(19) United States
(12) Patent Application Publication
(10) Pub. No.: US 2023/0083648 A1
(43) Pub. Date: Mar. 16, 2023

(54) CO2 EMISSION AMOUNT CALCULATION APPARATUS AND RECORING MEDIUM

(71) Applicant: TOYOTA JIDOSHA KABUSHIKI KAISHA, Aichi-ken (JP)

(72) Inventors: Masanori SHIMADA, Susono-shi (JP); Koichi UEDA, Susono-shi (JP); Naoto SUZUKI, Fujinomiya-shi (JP); Yoshihiro SAKAYANAGI, Mishima-shi (JP)

(21) Appl. No.: 17/858,073

(22) Filed: Jul. 6, 2022

(30) Foreign Application Priority Data
Sep. 14, 2021 (JP) 2021-149765

(51) Int. Cl. G05D 1/02; G01N 33/00

(52) U.S. Cl. CPC G05D 1/0212; G01N 33/004; G05D 2201/0213

(57) ABSTRACT

A CO₂ emission amount calculation apparatus includes a processor to search for a route from a departure place of a movable object to a destination, calculate, in response to re-routing of the movable object on the route searched for, a first CO₂ emission amount that the movable object emits in a route before the re-routing and a second CO₂ emission amount that the movable object emits in a route after the re-routing, and calculate, with the first CO₂ emission amount and the second CO₂ emission amount, a CO₂ emission amount that the movable object emits over an entire route from the departure place to the destination.

### CROSS-REFERENCE TO RELATED APPLICATION(S)

The present application claims priority to and incorporates by reference the entire contents of Japanese Patent Application No. 2021-149765 filed in Japan on Sep. 14, 2021.

### BACKGROUND

The present disclosure relates to a CO₂ emission amount calculation apparatus and a recording medium storing a CO₂ emission amount calculation program.

Japanese Laid-open Patent Publication No. 2010-127690 discloses a technology in which the CO₂ emission amount to be emitted from the internal combustion engine of a vehicle in each section from the departure place to the destination is calculated for searching for the CO₂ minimization route in which the total CO₂ emission amount from the departure place to the destination is minimum.

In calculation of the CO₂ emission amount to be emitted in a route from the departure place to the destination, a route change (re-routing) may occur, for example, due to a traffic jam or the choice of a wrong road. Thus, traveling is not necessarily performed along the first expected route. Thus, required has been a technology enabling proper calculation of the CO₂ emission amount from the departure place to the destination even in a case where re-routing has occurred.

### SUMMARY

There is a need for providing a CO₂ emission amount calculation apparatus and a CO₂ emission amount calculation program that enable proper calculation of the CO₂ emission amount from the departure place to the destination even in a case where re-routing has occurred.

According to an embodiment, a CO₂ emission amount calculation apparatus includes a processor to search for a route from a departure place of a movable object to a destination, calculate, in response to re-routing of the movable object on the route searched for, a first CO₂ emission amount that the movable object emits in a route before the re-routing and a second CO₂ emission amount that the movable object emits in a route after the re-routing, and calculate, with the first CO₂ emission amount and the second CO₂ emission amount, a CO₂ emission amount that the movable object emits over an entire route from the departure place to the destination.

According to an embodiment, a recording medium storing a CO₂ emission amount calculation program for causing a processor to: search for a route from a departure place of a movable object to a destination; calculate, in response to re-routing of the movable object on the route searched for, a first CO₂ emission amount that the movable object emits in a route before the re-routing and a second CO₂ emission amount that the movable object emits in a route after the re-routing; and calculate, with the first CO₂ emission amount and the second CO₂ emission amount, a CO₂ emission amount that the movable object emits over an entire route from the departure place to the destination.

### BRIEF DESCRIPTION OF THE DRAWINGS

FIG. 1 is a block diagram of the configuration of a CO₂ emission amount calculation apparatus according to an embodiment; and

FIG. 2 is a flowchart of an exemplary processing procedure of a CO₂ emission amount calculation method to be performed by the CO₂ emission amount calculation apparatus according to the embodiment.

### DETAILED DESCRIPTION

A CO₂ emission amount calculation apparatus and a CO₂ emission amount calculation program according to an embodiment of the present disclosure will be described with reference to the drawings. Note that the constituent elements in the following embodiment include constituent elements easily replaceable by those skilled in the art and substantially the same constituent elements.

CO₂ Emission Amount Calculation Apparatus

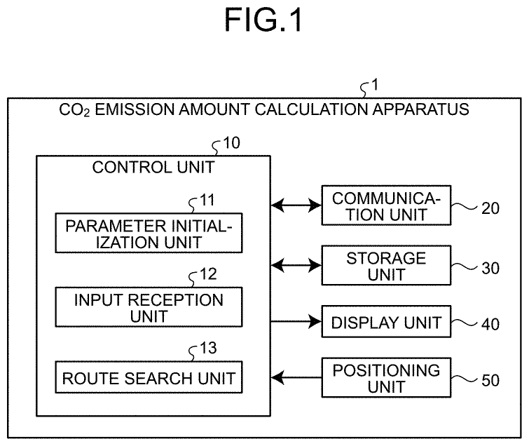

The CO₂ emission amount calculation apparatus according to the embodiment will be described with reference to FIG. 1. The CO₂ emission amount calculation apparatus is used in order to calculate the CO₂ emission amount when a movable object travels from the departure place to the destination.

A movable object to which the CO₂ emission amount calculation apparatus according to the embodiment is applied is, for example, a vehicle that travels along a set route. Examples of such a vehicle include an engine vehicle, a hybrid electric vehicle (HEV), a plug-in hybrid electric vehicle (PHEV), a fuel cell electric vehicle (FCEV), and a battery electric vehicle (BEV).

As illustrated in FIG. 1, a CO₂ emission amount calculation apparatus 1 includes a control unit 10, a communication unit 20, a storage unit 30, a display unit 40, and a positioning unit 50. Note that the CO₂ emission amount calculation apparatus 1 may be mounted on a movable object, such as a vehicle, or a server apparatus capable of communicating with a movable object. In the present embodiment, the CO₂ emission amount calculation apparatus 1 mounted on a movable object will be described.

Specifically, the control unit 10 includes a processor, such as a central processing unit (CPU), a digital signal processor (DSP), a field-programmable gate array (FPGA), or a graphics processing unit (GPU), and a memory (main storage unit) including a random access memory (RAM) and a read only memory (ROM).

The control unit 10 executes various types of programs to have centralized control over the operations of various types of constituent elements mounted on the CO₂ emission amount calculation apparatus 1. Due to execution of various types of programs, the control unit 10 functions as a parameter initialization unit 11, an input reception unit 12, and a route search unit 13.

The parameter initialization unit 11 initializes and sets various types of parameters for calculation of a CO₂ emission amount by the route search unit 13, to be described below. For example, parameters to be initialized and set by the parameter initialization unit 11 are as follows.

The weight M of the movable object [kg]

Travel resistance function Frl(V) [N]

CO₂ conversion factor R_{fuel} [kg/kg]

Efficiency function η_{eng}

External power consumption Et

Gravitational acceleration g [m/s²]

Here, the travel resistance function Frl(V) corresponds to a vehicle speed function and can be given, for example, by the following Expression (1) including coefficients a, b, and c.

Frl(V)=a×V²+b×V+c (1)

The external power consumption corresponds to power consumption, for example, due to the auxiliary machinery or air conditioner of the movable object. As the external power consumption, a previously set fixed value or the current value is used. Note that, in a case where a change in power consumption in the future can be predicted, for example, with a learning model, the predicted power consumption may be used.

As the efficiency function η_{eng}, a function for the power E[i] that the movable object needs to travel or a fixed value of 1 or less is used.

The input reception unit 12 receives a route search condition as input from a user (e.g., the driver). Examples of such a route search condition include the current date and time, the departure place, the destination, the presence or absence of priority on distance, the presence or absence of priority on arrival time, and the presence or absence of priority on CO₂ emission amount.

The input reception unit 12 displays, on the display unit 40, for example, a tab for selection of the current date and time, a tab or box for input of the departure place, a tab or box for input of the destination, a distance priority button, an arrival-time priority button, and a CO₂-emission-amount priority button. Then, on the basis of a selection by the user (e.g., a touch operation), the input reception unit 12 receives input. Note that the current date and time may be automatically input on the basis of a clock mounted on the movable object or time information acquired from a global positioning system (GPS) satellite. The departure place may be automatically input on the basis of positional information on the movable object acquired from a GPS satellite.

The route search unit 13 searches for a route from the departure place of the movable object to the destination, on the basis of any route search condition received as input by the input reception unit 12. The route search unit 13 displays, on the display unit 40, for example, a route search start button. Then, on the basis of a selection by the user (e.g., a touch operation), the route search unit 13 starts route search processing. Note that the route search unit 13 may search for one route or a plurality of routes (route candidates) from the set departure place to the set destination.

In response to re-routing of the movable object on the route already searched for, the route search unit 13 re-searches for a route from the current position of the movable object to the destination. Note that the “re-routing” means that the movable object deviates from the route previously searched for and set (makes a change in route), for example, by reason of a traffic jam or the choice of a wrong road. Specifically, the route search unit 13 re-searches for a route from the current position of the movable object acquired by the positioning unit 50 to the destination input at the time of the first route searching. The route search unit 13 may re-search for one route or a plurality of routes (route candidates) from the current position of the movable object to the destination.

In the route search processing of the route search unit 13, for example, Dijkstra's algorithm is used. In the route search processing with Dijkstra's algorithm, the i-th road searched for from the starting point (departure place) to the end point (destination) is subjected to route searching, for example, with the following information.

Travel time T[i] [s]

Road gradient Grad[i] [m/m]

Road length L[i] [m]

Here, the travel time T[i] may vary, depending on the date and time, even on the same road. Thus, as given by the following Expressions (2) and (3), the average vehicle speed V[i] [m/s] and the power E[i] [kW] required in traveling can be calculated per road. Note that, in a case where the movable object (e.g., a vehicle) performs no regeneration, the following Expression (3) satisfies E[i]≥0.

V[i]=L[i]/T[i] (2)

E[i]={(V[i]²−V[i−1]²)/2+M×g×Grad[i]×L[i]+Frl(V[i])×L[i]}/T[i]+Et[i] (3)

The route search unit 13 displays, on the display unit 40, routes (route candidates) searched for (re-searched for) as above. Note that the route search unit 13 may store as necessary a route search result (route re-search result) into the storage unit 30.

Here, in addition to the above route search processing, the route search unit 13 performs, per route searched for, calculation processing for the CO₂ emission amount that the movable object emits over the entire route from the departure place to the destination. On the basis of the parameters previously initialized and set by the parameter initialization unit 11, the power E[i], and the travel time T[i], the route search unit 13 calculates the fuel consumption Fuel[i] in accordance with the following Expression (4). Note that the fuel consumption Fuel[i] is not the amount of fuel actually consumed by the movable object but just a predicted value.

Fuel[i]=η_{eng}(E[i])×T[i] (4)

Next, the route search unit 13 calculates the CO₂ emission amount CO₂[i] in accordance with the following Expression (5).

CO₂[i]=Fuel[i]×R_{fuel}CO₂ (5)

Note that the CO₂ emission amount CO₂[i] calculated with the above Expression (5) is not a value calculated with account taken of the amount of energy actually consumed during traveling of the movable object (e.g., the fuel consumption) but a predicted value calculated before traveling of the movable object. In the present embodiment, a predicted value for the CO₂ emission amount of the movable object calculated in advance before traveling of the movable object as above is defined as the “advance-prediction CO₂ emission amount”.

In response to re-routing of the movable object on the route already searched for, in addition to route re-search processing, the route search unit 13 performs, per route re-searched for, calculation processing for the advance-prediction CO₂ emission amount over the entire route from the departure place to the destination.

In this case, the route search unit 13 calculates a first CO₂ emission amount that the movable object emits in the route before re-routing (route searched for first) (CO₂ emission amount before re-routing) and a second CO₂ emission amount that the movable object emits in the route after re-routing (route re-searched for) (CO₂ emission amount after re-routing). Note that the first CO₂ emission amount is required to be part of the CO₂ emission amount calculated on the basis of the route searched for first. Then, with the first CO₂ emission amount and the second CO₂ emission amount, namely, with addition of the second CO₂ emission amount to the first CO₂ emission amount, the route search unit 13 calculates the advance-prediction CO₂ emission amount that the movable object emits over the entire route from the departure place to the destination.

In addition to the advance-prediction CO₂ emission amount, the route search unit 13 may calculate a later-prediction CO₂ emission amount. The “later-prediction CO₂ emission amount” is a predicted value for the CO₂ emission amount that the movable object emits and is calculated by accumulation of the advance-prediction CO₂ emission amount after traveling of the movable object (or during traveling of the movable object) along the route previously searched for. That is, the later-prediction CO₂ emission amount is calculated by accumulation of the advance-prediction CO₂ emission amount corresponding to the route along which the movable object has already traveled, in the previously calculated advanced-prediction CO₂ emission amount.

Here, even in a case where a new route is re-searched for in response to re-routing of the movable object, the route search unit 13 calculates the later-prediction CO₂ emission amount by accumulating the advance-prediction CO₂ emission amount corresponding to the route along which the movable object has already traveled, in the advance-prediction CO₂ emission amount calculated after re-routing.

In addition to the advance-prediction CO₂ emission amount and the later-prediction CO₂ emission amount, the route search unit 13 may calculate a later-estimation CO₂ emission amount. The “later-estimation CO₂ emission amount” is an estimated value for the CO₂ emission amount that the movable object emits and is calculated on the basis of the amount of energy actually consumed during traveling of the movable object (e.g., the fuel consumption) after traveling of the movable object along the route previously searched for.

For example, in a case where the movable object is a vehicle including only an internal combustion engine as the power source, an electronic control unit (ECU) in the vehicle detects the actual fuel consumption per unit of time [kg/s]. Then, the route search unit 13 calculates the later-estimation CO₂ emission amount with addition of the actual fuel consumption multiplied by the CO₂ conversion factor R_{fuel} [kg/kg]. Thus, as the CO₂ emission amount that the movable object emits, used can be the later-estimation CO₂ emission amount higher in accuracy than the later-prediction CO₂ emission amount.

In addition to the routes (route candidates) searched for (re-searched for), the route search unit 13 displays, on the display unit 40, at least one of the advance-prediction CO₂ emission amount, the later-prediction CO₂ emission amount, and the later-estimation CO₂ emission amount calculated as above. Note that the route search unit 13 may store as necessary the advance-prediction CO₂ emission amount, the later-prediction CO₂ emission amount, and the later-estimation CO₂ emission amount into the storage unit 30.

Here, for example, as described in (1) to (3) below, the route search unit 13 may display, on the display unit 40, a result of comparison between any two of the advance-prediction CO₂ emission amount, the later-prediction CO₂ emission amount, and the later-estimation CO₂ emission amount.

(1) Display of Advance-Prediction CO₂ Emission Amount and Later-Prediction CO₂ Emission Amount on Display Unit 40

Display of the advance-prediction CO₂ emission amount and the later-prediction CO₂ emission amount on the display unit 40 enables comparison between the CO₂ emission amount to the destination expected at the departure place and the CO₂ emission amount expected on the basis of the route along which the movable object has actually traveled. This enables mainly visual quantification of variations in CO₂ emission amount due to re-routing.

(2) Display of Advance-Prediction CO₂ Emission Amount and Later-Estimation CO₂ Emission Amount on Display Unit 40

Display of the advance-prediction CO₂ emission amount and the later-estimation CO₂ emission amount on the display unit 40 enables comparison between the CO₂ emission amount to the destination expected at the departure place and the CO₂ emission amount estimated to have been actually emitted from the movable object in the route along which the movable object has actually traveled. This enables mainly visual quantification of variations in CO₂ emission amount due to re-routing and variations in CO₂ emission amount due to the way of running of the movable object (user's driving technique).

(3) Display of Later-Prediction CO₂ Emission Amount and Later-Estimation CO₂ Emission Amount on Display Unit 40

Display of the later-prediction CO₂ emission amount and the later-estimation CO₂ emission amount on the display unit 40 enables comparison between the CO₂ emission amount expected on the basis of the route along which the movable object has actually traveled and the CO₂ emission amount estimated to have been actually emitted from the movable object in the route along which the movable object has actually traveled. This enables mainly visual quantification of variations in CO₂ emission amount due to the way of running of the movable object (user's driving technique).

The communication unit 20 includes, for example, a local area network (LAN) interface board and a wireless communication circuit for wireless communication. For example, in a case where the CO₂ emission amount calculation apparatus 1 is achieved by a server apparatus capable of communicating with the movable object, the communication unit 20 communicates with the CO₂ emission amount calculation apparatus 1, to acquire, for example, a route search result or a result of calculation of a CO₂ emission amount from the CO₂ emission amount calculation apparatus 1.

The storage unit 30 includes recording media, such as an erasable programmable ROM (EPROM), a hard disk drive (HDD), and a removable medium. Examples of such a removable medium include a universal serial bus (USB) memory and disc recording media, such as a compact disc (CD), a digital versatile disc (DVD), and a Blu-ray (registered trademark) disc (BD). The storage unit 30 can store, for example, an operating system (OS), various types of programs, various types of tables, and various types of databases.

As necessary, the storage unit 30 stores, for example, the various types of parameters set by the parameter initialization unit 11, any route search condition received by the input reception unit 12, a route search result from searching by the route search unit 13, and any CO₂ emission amount calculated by the route search unit 13.

The display unit 40 is achieved, for example, by a liquid crystal display (LCD) or an organic EL display (OLED). The display unit 40 may be achieved, for example, by the display of a car navigation system mounted on the movable object. The display unit 40 may be achieved, for example, by a touch panel display having an input function of receiving an operation due to a finger of a user (e.g., the driver) or an operation due to a pen and a display function of displaying various types of information on the basis of the control of the control unit 10.

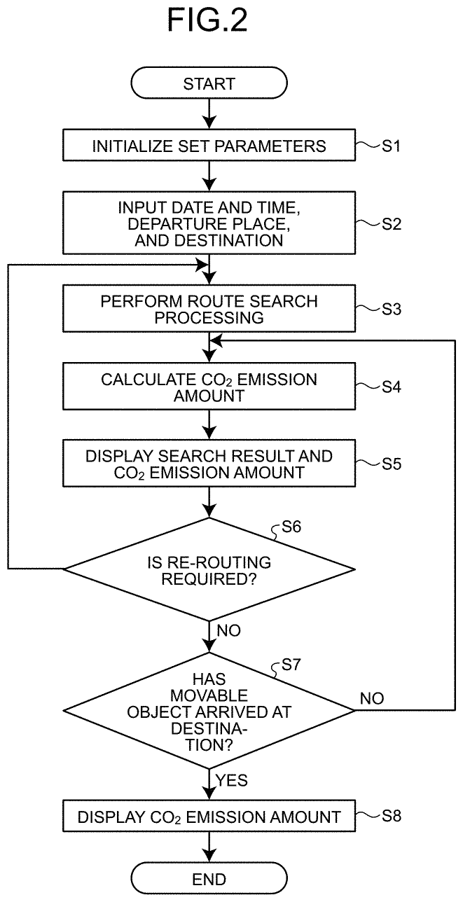

The positioning unit 50 receives radio waves from GPS satellites, to detect positional information and time information on the movable object. Note that the method of detecting positional information on the movable object is not limited to a method with GPS satellites. For example, used may be a method with light detection and ranging or laser imaging detection and ranging (LiDAR) and a 3D map in combination. The positional information on the movable object detected by the positioning unit 50 is used, for example, in route search processing of the route search unit 13 (Step S3 of FIG. 2 to be described below), in determination of whether re-routing is required (Step S6 of FIG. 2), and in determination of whether the movable object has arrived at the destination (Step S7 of FIG. 2).

CO₂ Emission Amount Calculation Method

An exemplary processing procedure of a CO₂ emission amount calculation method to be performed by the CO₂ emission amount calculation apparatus 1 according to the embodiment will be described with reference to FIG. 2.

First, the parameter initialization unit 11 initializes various types of parameters for calculation of a CO₂ emission amount (Step S1). Next, the input reception unit 12 receives, as input, route search conditions such as the current date and time, the departure place, and the destination (Step S2).

Next, on the basis of the route search conditions as input received by the input reception unit 12, the route search unit 13 performs route search processing from the departure place of the movable object to the destination (Step S3). Next, the route search unit 13 performs, per route searched for, calculation processing for the CO₂ emission amount that the movable object emits (Step S4). Note that, in Step S4, the advance-prediction CO₂ emission amount is calculated before traveling of the movable object and the later-prediction CO₂ emission amount and the later-estimation CO₂ emission amount are calculated during/after traveling of the movable object.

Next, the route search unit 13 displays, on the display unit 40, a route search result and the CO₂ emission amount (Step S5). Note that, in Step S5, the route search unit 13 displays, on the display unit 40, the advance-prediction CO₂ emission amount.

Next, the route search unit 13 determines whether re-routing is required (Step S6). In Step S6, for example, on the basis of the positional information on the movable object detected by the positioning unit 50, determination of whether re-routing is required is performed. That is, in a case where the current position of the movable object is out of the route searched for in Step S3, it is determined that re-routing is required, otherwise, it is determined that no re-routing is required.

In a case where it is determined in Step S6 that no re-routing is required (No in Step S6), the route search unit 13 determines whether the movable object has arrived at the destination (Step S7). In Step S7, for example, on the basis of the positional information on the movable object detected by the positioning unit 50, determination of whether the movable object has arrived at the destination is performed. That is, in a case where the distance between the current position of the movable object and the destination is a certain value or less, it is determined that the movable object has arrived at the destination, otherwise, it is determined that the movable object has not arrived at the destination.

In a case where it is determined in Step S7 that the movable object has arrived at the destination (Yes in Step S7), the route search unit 13 displays the CO₂ emission amount on the display unit 40 (Step S8), and then completes the present processing. Note that, in Step S8, the route search unit 13 displays, on the display unit 40, at least one of the advance-prediction CO₂ emission amount, the later-prediction CO₂ emission amount, and the later-estimation CO₂ emission amount.

Here, in a case where it is determined in Step S6 that re-routing is required (Yes in Step S6), the route search unit 13 goes back to the processing in Step S3. Then, in Step S3, the route search unit 13 re-searches for a route from the current position of the movable object to the destination, followed by the processing of Step S4.

In a case where it is determined in Step S7 that the movable object has not arrived at the destination (No in Step S7), the route search unit 13 goes back to the processing in Step S4 and performs the processing from Step S4.

According to the CO₂ emission amount calculation apparatus and the CO₂ emission amount calculation program according to the embodiment described above, searching for a route from the departure place to the destination is performed simultaneously with calculation of the CO₂ emission amount to be emitted in the route. In response to re-routing on the route searched for, the CO₂ emission amount over the entire route from the departure place to the destination is calculated with the CO₂ emission amount in the route before re-routing and the CO₂ emission amount in the route after re-routing.

Therefore, even in a case where re-routing has occurred, the CO₂ emission amount calculation apparatus and the CO₂ emission amount calculation program according to the embodiment enable proper calculation of the CO₂ emission amount from the departure place to the destination.

According to the CO₂ emission amount calculation apparatus and the CO₂ emission amount calculation program according to the embodiment, calculation and presentation of the advance-prediction CO₂ emission amount and the later-prediction CO₂ emission amount enable a user (e.g., the driver) to grasp the CO₂ emission amount to be emitted in the travel route and to grasp how much reduction can be made in CO₂ emission amount, for example, depending on other route search conditions or re-routing.

According to the CO₂ emission amount calculation apparatus and the CO₂ emission amount calculation program according to the embodiment, calculation of the later-estimation CO₂ emission amount and presentation of the later-estimation CO₂ emission amount together with the advance-prediction CO₂ emission amount or the later-prediction CO₂ emission amount enable a user (e.g., the driver) to grasp how much reduction can be made in CO₂ emission amount depending on the way of user's driving.

According to an embodiment, even in a case where re-routing has occurred, the CO₂ emission amount from the departure place to the destination can be properly calculated.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A CO₂ emission amount calculation apparatus comprising
a processor configured to
search for a route from a departure place of a movable object to a destination,
calculate, in response to re-routing of the movable object on the route searched for, a first CO₂ emission amount that the movable object emits in a route before the re-routing and a second CO₂ emission amount that the movable object emits in a route after the re-routing, and
calculate, with the first CO₂ emission amount and the second CO₂ emission amount, a CO₂ emission amount that the movable object emits over an entire route from the departure place to the destination.

2. The CO₂ emission amount calculation apparatus according to claim 1, further comprising
a display, wherein
the processor is configured to display, on the display, the route searched for and the CO₂ emission amount that the movable object emits over the entire route from the departure place to the destination.

3. The CO₂ emission amount calculation apparatus according to claim 2, wherein
the processor is further configured to
calculate, before traveling of the movable object along the route, an advance-prediction CO₂ emission amount, based on a plurality of parameters regarding the movable object, the plurality of parameters being previously set,
calculate, after traveling of the movable object along the route, a later-prediction CO₂ emission amount by accumulating the advance-prediction CO₂ emission amount,
calculate, after traveling of the movable object along the route, a later-estimation CO₂ emission amount, based on an amount of energy consumed during traveling of the movable object, and
display, on the display, at least one of the advance-prediction CO₂ emission amount, the later-prediction CO₂ emission amount, and the later-estimation CO₂ emission amount.

4. A non-transitory computer-readable recording medium storing a CO₂ emission amount calculation program for causing a processor to:
search for a route from a departure place of a movable object to a destination;
calculate, in response to re-routing of the movable object on the route searched for, a first CO₂ emission amount that the movable object emits in a route before the re-routing and a second CO₂ emission amount that the movable object emits in a route after the re-routing; and
calculate, with the first CO₂ emission amount and the second CO₂ emission amount, a CO₂ emission amount that the movable object emits over an entire route from the departure place to the destination.

5. The non-transitory computer-readable recording medium storing the CO₂ emission amount calculation program according to claim 4 for further causing the processor to display, on a display included in a CO₂ emission amount calculation apparatus, the route searched for and the CO₂ emission amount that the movable object emits over the entire route from the departure place to the destination.

6. The non-transitory computer-readable recording medium storing the CO₂ emission amount calculation program according to claim 5 for further causing the processor to:
calculate, before traveling of the movable object along the route, an advance-prediction CO₂ emission amount, based on a plurality of parameters regarding the movable object, the plurality of parameters being previously set;
calculate, after traveling of the movable object along the route, a later-prediction CO₂ emission amount by accumulating the advance-prediction CO₂ emission amount;
calculate, after traveling of the movable object along the route, a later-estimation CO₂ emission amount, based on an amount of energy consumed during traveling of the movable object; and
display, on the display, at least one of the advance-prediction CO₂ emission amount, the later-prediction CO₂ emission amount, and the later-estimation CO₂ emission amount.
